(19) United States
(12) Patent Application Publication
(10) Pub. No.: US 2024/0009227 A1
(43) Pub. Date: Jan. 11, 2024

(54) USE OF A HEPARIN COMPOSITION IN THE TREATMENT OF VIRAL LUNG DISEASES, ACUTE AND/OR CHRONIC LUNG DISEASES BY SOFT MIST INHALER OR VIBRATION MESH TECHNOLOGY NEBULIZER THROUGH INHALATION ROUTE

(71) Applicant: ISTANBUL UNIVERSITESI REKTORLUGU, Fatih/Istanbul (TR)

(72) Inventors: Ayca Yildiz PEKOZ, Fatih/Istanbul (TR); Mustafa ERELEL, Fatih/Istanbul (TR)

(21) Appl. No.: 18/041,642

(22) PCT Filed: Jun. 18, 2021
(86) PCT No.: PCT/TR2021/050630

§ 371 Date: Feb. 14, 2023

(30) Foreign Application Priority Data
Aug. 14, 2020 (TR) 2020/12816
Jan. 14, 2021 (TR) 2021/00552

(51) Int. Cl. A61K 31/727; A61K 9/00; A61K 31/047; A61K 31/198; A61K 33/14; A61K 31/375; A61K 31/573; A61P 31/14

(52) U.S. Cl. CPC A61K 31/727; A61K 9/0078; A61K 31/047; A61K 31/198; A61K 33/14; A61K 31/375; A61K 31/573; A61P 31/14

(57) ABSTRACT

The present invention relates to the administration of heparin or its derivatives, which are anticoagulant, especially low molecular weight heparin (LMWH) in the treatment of especially COVID-19, viral lung diseases, acute and/or chronic lung diseases by means of soft mist inhaler or vibrating mesh technology (VMT) nebulizer through inhalation route. In the present invention, heparin and its derivatives may be administered by means of the passive vibrating mesh nebulizer or active vibrating mesh nebulizer. Anticoagulant heparin or its derivatives reach the lungs efficiently and quickly, and local pulmonary administration is performed such that it provides an effective treatment. Since the drug is targeted directly to the lungs without getting into systemic circulation via local (direct) administration, its concentration is higher at the application region, thereby reducing the side effects and costs per application of the drug, and increasing its efficacy. The pulmonary route is an optimal route of administration for drugs that are poorly absorbed or quickly metabolized through the oral route.

### TECHNICAL FIELD OF THE INVENTION

The present invention relates to the administration of heparin or its derivatives, which are anticoagulant, especially low molecular weight heparin (LMWH) in the treatment of especially COVID-19, viral lung diseases, acute and/or chronic lung diseases by means of soft mist inhaler or vibrating mesh technology (VMT) nebulizer through inhalation route. In the present invention, heparin and its derivatives may be administered by means of the passive vibrating mesh nebulizer or active vibrating mesh nebulizer.

### STATE OF THE ART

Coronaviruses (CoV) are a large family of viruses that cause diseases ranging from the common cold to more serious diseases such as Middle East Respiratory Syndrome (MERS-CoV) and Severe Acute Respiratory Syndrome (SARS-CoV). Coronaviruses are single-stranded, positive-polarity, enveloped RNA viruses. They have rod-like extensions (protrusions) on their surfaces. The Latin equivalent of the crown-like structure formed by these protrusions is “corona”, and based on this, these viruses were named Coronavirus (coronavirus, crowned virus). Coronaviruses are classified into four main genera alpha-, beta-, gamma- and delta coronaviruses. They can be detected in humans, domestic and wild animals (bat, camel, pig, cat, dog, rodent and poultry, etc.). Human coronaviruses were first identified in the 1960s. Today, there are seven coronaviruses known to have infection factors in humans. 229E (Alpha coronavirus), NL63 (Alpha coronavirus), (Beta coronavirus), and HKU1 (Beta coronavirus) are the coronaviruses that are the most common infectious factors in humans and affect the upper and lower respiratory tract. Three other human coronaviruses have been identified recently, and they are SARS-CoV, MERS-CoV, and lastly SARS-CoV-2. SARS-CoV virus has been identified in 2002 in China. It causes Severe Acute Respiratory Syndrome (SARS). The epidemic caused the death of 774 people worldwide. MERS-CoV emerged in Saudi Arabia in 2012 and was named Middle East Respiratory Syndrome virus (MERS), it spread to 24 countries and caused more than 1000 cases and around 400 deaths. SARS-CoV-2, on the other hand, is an infectious and extremely pathogenic coronavirus that started in Wuhan, Hubei province of China in the last days of December 2019, and caused pneumonia in humans, an epidemic of severe respiratory tract infection, and subsequently spread first across the country, and then all over the world. The epidemic was initially detected in people who are in the seafood and animal market in this region; however, it later spread from person to person and spread to other cities in Hubei province, especially Wuhan, to other provinces of the People's Republic of China, and to other countries in the world due to the interaction between people and travels. Since December 2019, when the virus first appeared, the world population has almost quarantined globally, and it caused a global economic slowdown. Since there is no full treatment, how long this quarantine will last and its negative effect on people and economies is unpredictable; it caused the death of more than 3.8 million people until July 2021, and it is stated that this number will exceed millions until herd immunity is achieved.

This new viral respiratory disease caused by the SARS-CoV-2 virus and the most common symptoms thereof, which manifest themselves as high fever, cough, and respiratory distress (dyspnea, difficulty in breathing), has been defined as COVID-19 by the World Health Organization. The SARS-CoV-2 virus directly targets the lungs, and lung destruction begins in a short time as 5 days. Patients generally die due to respiratory failure. At present, a drug capable of completely treating COVID-19 clinically is not available. The drugs currently used are antivirals, cytokine inhibitors, and antibody administration methods, which are used in the palliative treatments of previous viruses.

COVID-19 is transmitted by means of coughing/sneezing of sick individuals and inhaling droplets spread in the environment. People might get infected with the virus in case they touch their face, eyes, nose, or mouth without washing/disinfecting them after touching surfaces contaminated with respiratory particles of sick people. For this reason, touching the eyes, nose, or mouth with dirty hands during this epidemic carries a great risk. The incubation period of the SARS-CoV-2 coronavirus is between 2 days and 14 days, and milder complaints (such as fever, sore throat, weakness) are observed in the first few days of the disease, and after then, symptoms, which manifest themselves as cough and difficulty in breathing (dyspnea) are observed, and conditions of patients usually become severe enough to apply to the hospital after 7 days. In consideration of the data obtained, the virus contains a higher risk of causing severe disease for people of advanced age (65 years and older) and accompanying disease (asthma, diabetes, heart disease, etc.). Some of the people infected with SARS-CoV-2 coronavirus survive the disease mildly and do not show symptomatic indications, however, since said individuals are the carriers, they carry the disease to the people they get in contact with. Carrier patients generally are children and young individuals. Although the current data indicate that the mortality rate of the disease is around 2% but said information may differ depending on the changes that may occur in the genetic structure of the virus. In severe cases, pneumonia, severe acute respiratory tract infection, severe respiratory failure, kidney failure, and even death may occur.

It is known that viral infection affects the respiratory system and cardiac system with the pathogenesis of the SARS-CoV-2 virus starting in the body. Data obtained from the cohort and the autopsies of deceased patients indicate that people infected with the SARS-CoV-2 virus develop a coagulopathic profile. A multicenter retrospective cohort study conducted in the People's Republic of China has included 191 adult patients who were proven to have COVID-19 through laboratory data. Coagulopathy was observed in 50% of patients who died. The rate of coagulopathy with sepsis complications was recorded as 70% in patients who died. In addition, coagulative abnormalities were observed in patients infected with COVID-19, but it has also been stated that these are not the typical disseminated intravascular coagulation (DIC) observed in sepsis. Furthermore, lung microthrombi formation was also confirmed in patients who were subjected to autopsy.

In addition to thrombus formation in patients infected with the SARS-CoV-2 virus, it is assumed that the procoagulant and anticoagulant state that is observed during infection triggers the balance disorder between immune and non-immune cells, and also triggers a thrombus formation. The endothelium plays a critical role in maintaining body homeostasis and it is known that viral infections will disrupt the integrity of the endothelium, and it causes a possible risk of hematopathology. Additionally, it is thought that von Willebrand factor elimination, T-like receptor activation, and tissue factor pathway activation that is induced as a result of viral infection play a role in the coagulant cascade together, and this effect causes cross-linked fibrin coagulation. Each physiological response for excessive activation of the coagulant cascade required for the destruction of these clots is responsible for the procoagulant D-dimer factor. Following antigen recognition, platelets are activated in addition to D-dimer, thereby allowing white blood cells to coordinate for the purpose of removing pathogens and forming coagulation. As a result, immune cells, platelets, and endothelial cells play a role in the formation of the coagulopathic profile in viral infection. In addition to this clinical picture, it should be taking into consideration that the picture of venous thromboembolism will also constitute an additional reason in favor of coagulation since COVID-19 patients are on bed rest for a long time.

COVID-19 disease that is described in detail above disrupts the coagulation pathway and causes a severe course of the disease. Therefore, heparin is generally preferred by the healthcare professional as the first-choice anticoagulant administration in the treatment of said disease. Heparin is a highly sulfated glycosaminoglycan available in the mast cells of many mammals. The compound binds to immune response proteins such as coagulation factors, growth factors, cytokine, and chemokine by means of this acidic property. In case the anticoagulant mechanism of heparin is disclosed briefly; heparin binds to antithrombin (AT) and potentiates the actions of AT to inactivate factor Xa and prevent the conversion of prothrombin to thrombin, as well as prevent the conversion of fibrinogen to fibrin (1). Heparin also binds nonspecifically to various plasma proteins and endothelial cells resulting in an unpredictable dose-response relationship and low bioavailability after subcutaneous (SC) administration. Low molecular weight heparins (LMWHs) also bind AT and accelerate the activity of AT, but with a preferential, and longer-lasting effect on factor Xa. When compared to heparin, LMWHs are less able to inhibit the production of thrombin and bind to plasma proteins and endothelial cells less due to their decreased sized (2). This accounts for an 85-99% bioavailability when administered SC, more predictable anticoagulant response, less inter-patient variability, and longer duration of action than heparin (3). Today, doses of LMWH administered at once in the treatment of diseases are in the range of 4000 IU-10000 IU. The total daily dose, on the other hand, is currently used in the treatment of COVID-19 in doses up to 20000 IU.

A positive improvement is observed in prothrombin times of 99 patients out of 449 patients with severe course of the disease, who were administered anticoagulant heparin (especially low molecular weight heparin) parenterally for at least 7 days, thereby it was reported that a negative improvement is observed in mortality rate and platelet count in a retrospective study conducted in the People's Republic of China, where the SARS-CoV-2 virus was first detected.

Heparin, in addition to the anticoagulant effect thereof, has anti-inflammatory and immunomodulatory properties in the respiratory system. Studies conducted in recent years have indicated that unfractionated heparin (UFH) reduces endotoxin-induced pulmonary vascular escape and has anti-inflammatory activity. In addition to this, it has been indicated that heparin has an anti-asthmatic effect against specific and nonspecific stimulations due to inflammation in patients with asthma in case of bronchial hyperreactivity. It alleviates the bronchial hyperreactivity induced by histamine and leukotriene. Anionic heparin acts by binding to various pro-inflammatory cytotoxic proteins and neutralizing these proteins (4). It has also been indicated that heparin affects neutrophil chemotaxis and lymphocyte flow (5). Considering all these studies, it has been proven that heparin or its derivatives featuring anticoagulant properties may be administered through inhalation. It has been indicated that enoxaparin-sodium, which is low molecular weight heparin, reduces mast inflammatory mediators and eosinophils when administered by means of inhalation. It has been indicated that it causes interleukin-6 (IL6), interleukin-8 (IL8), and tumor necrosis factor-alpha (TNF-α) activation when the blood encounters a foreign surface during the bypass, and these inflammatory molecules are reduced in case heparin-coated materials are used. Moreover, this effect is directly proportional to the dose. The length of stay in the intensive care unit has also decreased for these patients.

Heparin also antagonizes histone. Histone is released from damaged cells and thereby causing histone damage in COVID-19 infection. Enkhbaatar et al. have indicated that nebulized heparin increases oxygenation and reduces pulmonary edema by acting with its histone antagonizing feature in smoke-induced lung injury (6).

Heparin has been used in the treatment of Acute Lung Injury (ALI) in recent years. ALI may stem from a variety of reasons. This results in refractory hypoxemia and difficulty in breathing. Vascular permeability increase, protein-containing substance exudation, and fibrin deposition stem from inflammatory mediator release in ALI. In the ALI presentation, 40-60% was accepted as mortality. In this context, an in vitro study conducted by Camprubi-Rimblas et al., in 2017 revealed that heparin used in a lung cell model simulating ALI significantly inhibited the NF-κB pathway. It has been indicated that said inhibition also reduces IL-6 and TNF-α levels in human lung macrophages. It has been noted that heparin significantly reduces IL-6, TNF-α, and monocyte chemo-induced protein-1 (MCP-1) levels in human alveolar Type II cell models. It has been observed that nebulized heparin reduces ALI symptoms via pro-coagulant and pro-inflammatory pathways in an in vivo study on a rat model with Acute Respiratory Disease Syndrome (ARDS) conducted by Chimenti et al., in 2017. Additionally, IL-6, TNF-α levels were significantly decreased in the same rats, and even a decrease in NF-κB expression in alveolar macrophages was reported (7). A randomized controlled observational study, which enrolled 60 patients diagnosed with severe ARDS, and in which nebulized heparin, streptokinase, and placebo were used, was conducted by Abdelaal Ahmed Mahmoud et al., in 2020. Accordingly, patients who administered 10,000 IU nebulized heparin every 4 hours showed a significant improvement in ARDS at the end of the 8th day. APTT and INR levels that are systemic coagulation markers did not change, and even a major level of hemorrhage or blood transfusion, which is a finding that favors the use of heparin was not observed (8). Most cases of COVID-19 had mild to moderate respiratory symptoms, and about 20% of said cases had severe respiratory diseases. Said respiratory diseases were mainly diagnosed as ALI and ARDS. Significant increases in inflammatory cytokine levels such as interleukin-2 (IL-2), IL-6, TNF-α, and MCP-1 have been reported in studies conducted in patients with severe disease. Said inflammatory cytokine level, which is known as “cytokine storm” is an indicator of the natural antiviral response of the body to viral RNA replication. The aforementioned viral replication also makes a downstream induction in the monocyte-macrophage infiltration and in inflammatory signal pathways like NF-κB and IRF3 which cause an increase in neutrophil count. In a holistic approach, said processes cause advanced respiratory complications that develop in patients infected with SARS-CoV-2. Various therapeutic strategies, which also include anticoagulants, are implemented by scientists to overcome ALI and ARDS profiles for COVID-19 or other cases.

Another positive effect of heparin observed on COVID-19 patients is that heparin binds to the spike protein of the SARS-CoV-2 virus, and also makes a downstream regulation in the expression of IL-6, which has an important role in the pathogenesis of COVID-19.

Systemic administration of commercially available unfractionated (UFHs) and fractionated low molecular weight heparins (LMWHs), and Ultra-Low Molecular-Weight heparins (ULMWHs) may cause a risk of hemorrhage as a result of anticoagulant properties thereof. Therefore, research has focused on targeting heparin by means of nebulization with the aim of controlling and preventing the aforementioned hemorrhage risk. It is indicated that the local effect of heparin is reduced in case heparin is administered systemically. Tests conducted on rabbits determined that it has increased the partial oxygen pressure and decreased the total protein content in the alveoli. Heparin further decreases the level of malondialdehyde (MDA), which is an indicator of endothelial damage, and in return, it also increases the amount of superoxide dismutase (SOD), which removes reactive oxygen products that cause ischemic damage, and glutathione peroxidase (GSH-Px), which protects from oxidative stress. A prospective study indicated that inhaled low molecular weight heparin required 10 times the dose that is administered subcutaneously in order to produce anticoagulation at the therapeutic level.

Jaques et al. mentioned inhaled heparin administration for the first time in 1976 in a scientific study published in the Lancet Journal with the title of “Intrapulmonary Heparin, A New Procedure for Anticoagulant Therapy”. Research performed 10-20 mg/m in heparin administration by means of using Devilbiss ultrasonic nebulizer. In the study, patients were asked to breathe slowly and deeply, and the practice continued for 90 minutes, including rest periods. The study compared the inhalation method with intravenous and subcutaneous methods. Consequently, it has indicated that inhaled heparin was significantly superior based on the level of side effects and the duration time of anticoagulant activity parameters.

Atz et al., in a study they conducted in 1998, researched the use of inhaled heparin together with nitric oxide for 4 months and younger infants with pulmonary hypertension. Consequently, it was revealed that nitric oxide that has antioxidant, antiproliferative, and antihypertensive effects plays an important role in the maintenance of primary hypertension treatment when used in combination with heparin that stimulates the development of smooth muscle and new vessels (9).

Dixon et al. evaluated the therapeutic effect of nebulized heparin in 16 patients in the early phase of acute lung injury thereof. In the study, it was observed that 4 doses of heparin did not cause a significant change in respiratory functions and systemic anticoagulant effect (10).

In another study, a preclinical and a clinical study were conducted by means of applying a treatment regimen including nebulized heparin, heparinoids, antithrombins, or fibrocytes. The indicated inhaled regimen has been proven to reduce morbidity without impairing coagulation and anticoagulation markers in preclinical and clinical studies (11). Chopra et al., in a study they conducted, indicated that aerosolized acetylcysteine/heparin application developed a clinically successful coagulopathy in a patient who burnt 87% of his/her body and who suffers from inhalation injury (12).

It has been demonstrated that inhaled heparin is capable of reducing sputum clearance and that it does not show any indication of hemorrhage or any other side effects when it is administered in 50.000 IU twice a day for two weeks to patients with cystic fibrosis (13). Although the effect of heparin on bronchial hyperreactivity is known, mechanisms of action thereof have not been fully resolved yet. A great number of in vitro, in vivo, preclinical, and clinical studies have indicated that the main function of heparin is to reduce mast sell degranulation and mechanisms that cause inflammation thereof, rather than its direct effect on smooth muscles (14-17). Heparin is a highly sulfate-containing glycosaminoglycan available in the mast cells of many mammals. It prevents coagulation with its acidic feature. Heparin, in addition to the anticoagulant effect thereof, also has anti-inflammatory and immunomodulatory properties. Fibrinolytic property of heparin and its derivatives; they also have the ability to effect by means of binding to immune response proteins such as growth factor, cytokine, and chemokine. In addition, heparin, which is a polyanionic protein, is a highly effective inhibitor for virus binding. Herpes simplex competes with the virus for binding to surface glycoproteins in the host cell in Zika virus infections. More importantly, it has been reported that heparin inactivates the virus and suppresses interleukin 6's by means of binding to the “spike proteins” of the virus in patients with extremely severe COVID-19. It antagonizes histone released from damaged lung cells in COVID-19 disease. Numerous preclinical and clinical studies have been published on the use of ‘inhaled’ heparin in lung diseases.

Tuinman et al. (2012) determined that the survival rate of patients increased in ALI dependent on smoke inhalation of nebulized heparin, and in preclinical studies, nebulized administration of heparin created the desired systemic coagulation effect without causing hemorrhage when it is compared to systemic administration (18).

Although there are ongoing clinical studies regarding the use of heparin and its derivatives in the treatment of COVID19 with a nebulizer, there is still no published clinical study data. One of said ongoing studies is the study protocol titled COVID-19 HOPE (Nebulized Heparin-N-acetylcysteine in COVID-19 Patients by Evaluation of Pulmonary Function) in the USA. In this study protocol conducted by Steven Quay et al., it is thought that the number of patients who require mechanical ventilation will decrease, and in some cases, this requirement will disappear completely when heparin is administered in combination with N-acetyl cysteine through the inhalation route in COVID-19 patients.

Except for the COVID-19 HOPE study, there are 38 ongoing clinical studies that analyze the anticoagulant activity in the treatment of COVID-19, and 30 of them use heparin and its derivatives as anticoagulants. These studies have preferred subcutaneous and intravenous administration routes, which are the conventional drug administration routes for the administration of heparin and its derivatives. Only one of these studies (Johns Hopkins University-based clinical research submitted on May 21, 2020, and started on Jun. 1, 2020) aims to compare and analyze the effects with the nebulized physiological saline application by means of using heparin substance as nebulized.

In the state of the art, the patent document numbered RU2269346C1 discloses a method for introducing pathogenic heparin into a part defined as the tracheobronchial tree of the patient in a dose of 700 IU/kg 3-6 times in 3-5 days for the treatment of tuberculosis. Here, the administration of heparin by means of inhalation or an endobronchial application is protected. Another patent application numbered U.S. Pat. No. 4,679,555A in the prior art discloses intrapulmonary administration of heparin sodium in the powder or fine powder form by means of a metered-dose inhaler containing a low boiling point chlorofluorocarbon group propellant. On the other hand, the patent application numbered US2002195101A1 in the state of the art discloses a stationary inhalation apparatus for administering therapeutic aerosols in an individually controlled manner. Said patent application also discloses the use of said stationary inhalation apparatus for aerosolized administration of low molecular weight heparin or a medicament in order to prevent thrombosis. Another patent application numbered CN109260181A in the prior art discloses pharmaceutical solutions in liquid form that are prepared by mixing a pH adjuster excipient, an isotonic excipient, and a surfactant in purified water together with the pharmaceutically acceptable salt of heparin, and suitable for subsequent application in atomized form. It has been indicated that said solutions can be used in the treatment of COPD, acute lung injury, and acute respiratory distress syndrome.

The US patent document titled “Aerosolization Device” numbered US 2014020680A1 in the state of the art discloses a nebulizer device that allows for producing an aerosol cloud containing a therapeutic agent therein and operates with a vibrating mesh system. Said patent document does not mention the therapeutic administration of a substance with anticoagulant properties, and the use of amikacin and vancomycin antibacterial substances as therapeutic agents, which are stated in the claims thereof in the indication of coronavirus.

The US patent document titled “Unit aerosol doses for anticoagulation” numbered U.S. Pat. No. 10,668,015B2 in the state of the art mentions inhaled administration of an active substance, which is an anticoagulant called argatroban, and which is a small molecule direct thrombin inhibitor prophylactically in Acute Coronary Syndrome.

Said patent document does not mention any anticoagulant agent except argatroban and mentions only prophylactic inhalation of argatroban substance only in Acute Coronary Syndrome, and the vibrating mash nebulizer device is not specifically emphasized and the use of anticoagulants is not mentioned.

Heparin is currently used parenterally in patients with COVID-19. However, parenteral use thereof causes the following limiting effects on the activity of heparin:
1) Parenteral administration of heparin reduces its local effect on the lungs in patients with COVID-19.
2) Parenteral administration of heparin produces systemic effects in the entire body. This causes undesirable hemorrhage in the entire body and some undesirable side effects. A prospective study indicated that inhaled low molecular weight heparin required a dose of 10 times the dose that is administered subcutaneously in order to produce anticoagulation at the therapeutic level. These data indicate the result of that local administration of heparin to the lungs will not affect the entire body systemically.

The choice of a drug that is used in the treatment of lung diseases (as in any organ or tissue) is primarily for the local treatment of said organ or tissue. Local treatment ensures the drugs to be used are effective only in the determined organ or tissue, and other parts of the body are not exposed to the drug systemically. The administration results in more effective and the side effects thereof are reduced by means of the local administration of the drug, although the active substances are applied in lower amounts. The effect of neutralizing the viral load of COVID-19 and preventing the virus from entering the cell by means of binding to spike proteins has been identified together with the anticoagulant and anti-inflammatory effect of heparin. This pharmacological feature of heparin indicates that its antiviral effect on COVID-19 will create a more effective and successful use when compared to parenteral administration in case it is administered locally to the lungs. Similarly, it is known that local treatment is more effective and successful in the treatment of other viral lung diseases compared to oral or parenteral applications.

COVID-19 pandemic necessitates dosage forms that may be formulated very quickly and technologies thereof. Inhalation devices used in the clinic are metered-dose inhaler (MDI), dry powder inhaler (DPI), nebulizers (Jet, ultrasonic, new type nebulizer (e.g. VMT and electronic), and soft mist inhalers). The use of MDI and DPI's are not very advantageous, especially for patients with severe respiratory distress, and involve many drawbacks (difficulty of use, inability to control their activity, risk of contamination). At this point, device selection becomes prominent. Standard nebulizers are not safe in COVID-19 patients due to common tidal breathing problems, wide distribution of droplets, distribution of patient saliva by the nebulizer, and posing a risk of infection for health care personnel. In practical terms, jet, ultrasonic, or electronic nebulizers cause distribution of the virus and pose a risk of infection, and they should not be preferred with regard to the wellness of health care personnel due to the fact that they cause physician and nurse deaths as observed in Italy and USA. Droplets scattered in breathing carry viruses and it is very important to minimize this risk during the treatment process. Therefore, choosing the right administration route and the right nebulizer is extremely important in the treatment of viral lung diseases including COVID-19 disease.

Soft mist inhaler (so named to describe aerosol production mechanisms and aerosol-cloud properties) is a non-pressure metered dose inhaler that uses microfluidic technology and features a measuring function that enables to delivery of different doses (19-20). In DPIs, the fine particle dose produced is highly dependent on the inspiratory stream of air and absolute lung capacity, which varies widely according to patients (19). On the other hand, soft mist inhalers provide many advantages in terms of lung accumulation and ease of use. Soft mist inhalers are active systems that do not require propellant, in other words, the energy required for aerosol production is supplied from the inhaler and is therefore independent of the inspiratory capacity of the patient (20). Soft mist inhalers provide many more advantages in terms of drug accumulation in the lungs and ease of use. The soft mist inhaler works with an active mechanism that does not require propellant; the energy required for aerosol production is provided from the inhaler itself. Thus, the soft mist inhaler is independent of the patient's respiratory capacity. The size range of the aerosol droplets released from the device is in the range of 2-6 micrometers and said aerosol droplets target the lungs. Another advantage of the soft mist inhaler is that dosing is performed by means of a syringe. The present parenteral form of the drug/active substances may be administered by integrating it into the soft mist inhaler without requiring an additional formulation step by means of said syringe system.

Dose-to-dose reproducibility of soft mist inhalers that enables delivering a drug in a solution form with a certain volume from a depot delivery system or a single-use dosage form is more consistent than dry powder inhalers, which release small amounts of suspension, and which are carried in powder. In soft mist inhalers, the drug is in dissolved form in solution; therefore, it is affected less by moisture ingress compared to dry powders, thus soft mist inhalers are suitable for use in areas with humid environmental conditions. The relatively low velocity and long spray time of the soft mist inhaler facilitate the inhalation of the aerosol in a reproducible manner. However, there is often a requirement that the drug is soluble and stable in the solution for the soft mist inhalers unless certain formulation technologies are not applied.

Historically, jet nebulizers have been the standard delivery system for aerosol drugs. They are relatively inefficient and require an external air source to operate. On the other hand, vibrating mesh technology was developed as an alternative to jet nebulizers. It is known that vibrating mesh technology nebulizers are more efficient than jet nebulizers and they do not require additional gas in the ventilator circuit. On the other hand, vibrating mesh nebulizers may be more sensitive to the contamination risk and device orientation and have precision electronic controls when compared to jet nebulizers. Vibrating mash technology (VMT) nebulizers provide many advantages with their consistent and improved aerosol production efficiency, fine particle fraction that can reach the peripheral lung, and nebulization capability in low residual volume and low drug volumes. VMT nebulizers are active systems that do not require propellant and that use micro-pump technology, and the energy required for aerosol production is provided from the inhaler in the physical mechanism. Therefore, drug delivery to the target region in the lungs is independent of the respiratory capacity of the patient. VMT nebulizers feature short processing times and silent operation. The pore size of VMT nebulizers may be optimized by adjusting the aerosol chamber and output rate for different drugs. VMT nebulizer, as a working principle, is based on the fact that thousands of holes on a membrane vibrate at the same time for hundreds of thousands of times per second, and the liquid that passes through these holes creates aerosol droplets with suitable size for targeting the drug to the lungs. The system control sensors detect if there is any liquid contact with the atomizing membrane, and allow the liquid to pass through thousands of holes created via precision laser by means of the vibrations in the resonant bending mode, thereby creating fine droplets having a narrower size distribution than the present systems. The membrane can be designed so as to yield droplets of a certain size that are suitable for the physical properties of the solution by means of changing the pore size of said membrane. The VMT nebulizer ensures that the dosing is carried out in a much better way since there is no aerosol escape unlike conventional nebulizers (jet or ultrasonic) by means of its system that fits into the mouth and that is developed for maskless use. In addition, the room contamination problem observed in the use of the classical type nebulizer in the treatment of COVID-19 is no longer a problem since the VMT nebulizer works in a closed system by means of its mouthpiece. In a VMT nebulizer, the drug is in dissolved form in solution, therefore, it is affected less by moisture ingress compared to dry powders, thus VMT nebulizers are suitable for use in humid environments. Another advantage of VMT nebulizers is that they facilitate the inhalation of aerosol in a reproducible manner by means of the long spraying time with the low velocity thereof. The drug to be applied in the vibrating mesh nebulizer is positioned on the concave side of the mesh and the mesh is vibrated at high frequency by using a piezoelectric actuator. This allows the drug to transform into a cloud consisting of small droplets that can be delivered from the bottom (convex) side of the mesh. In addition, the droplet size can be adjusted by means of said technology as mentioned above. In particular, geometrical changes can be performed to the mesh structure in order to provide a desired certain droplet size. The droplets may move away from the device under the force of gravity at low velocity due to the absence of atomization gas. In addition, the number of holes in the mesh and their placement on the mesh may also be customized.

The limitations and inadequacies of the available solutions in the current technique necessitated making an improvement for the effective treatment of especially COVID-19, viral lung diseases, acute lung diseases, and/or chronic lung diseases.

### BRIEF DESCRIPTION AND OBJECTS OF THE INVENTION

The present invention discloses the administration of heparin and its derivatives, which are anticoagulants, especially low molecular weight heparin (LMWH) for use in the treatment of symptoms caused by especially COVID-19, viral lung diseases, acute and/or chronic lung diseases by means of using soft mist inhaler or vibrating mesh technology (VMT) nebulizer through inhalation route, and compositions including heparin and its derivatives, effective dosage forms, and doses. In the invention, said anticoagulant substance is administered locally and directly to the lung through the pulmonary route. The pulmonary route is a suitable route for administering active substances with weaker absorption features than the oral route and with peptide-protein structures that are broken down in the stomach, or active substances that are rapidly metabolized. The pharmaceutical composition subject to the invention may contain an additional active substance and/or excipients as well as heparin or heparin derivatives.

The most important object of the present invention is to provide effective treatment of especially COVID-19, viral lung diseases, acute lung diseases, and/or chronic lung diseases. The present invention allows the active substance is administered locally (directly) to the lung in the treatment of viral lung diseases such that it has many advantages compared to the other administration routes (oral, parenteral, etc.), thereby, providing more effective treatment.

Another object of the present invention is to ensure that the drugs/active substances used in the treatment of especially COVID-19, viral lung diseases, acute lung diseases, and/or chronic lung diseases are effective with higher efficacy and minimize the side effects thereof. In the present invention, the drug efficacy increases, and side effects of the drug, which may occur systemically are reduced by means of its local administration compared to the oral and parenteral routes.

Yet another object of the present invention is to provide effective treatment of especially COVID-19, viral lung diseases, acute lung diseases, and/or chronic lung diseases by means of an application with high bioavailability. In the present invention, administration of heparin and its derivatives through the pulmonary route increases the bioavailability since the effect of liver first pass is eliminated. In addition, since the pass of macromolecular structures through the lungs is quite well, the effectiveness of the treatment is higher than the current administration methods.

Yet another object of the present invention is to treat the damage caused by the COVID-19 disease to the lungs. In the present invention, cases such as acute lung injury caused by SARS-CoV-2 virus in the lungs, bronchial hypersensitivity due to inflammation, thromboembolism, histone release from damaged lung cells, and histone damage in the lungs, ARDS, and hypoxemia associated therewith are treated by means of administration of heparin or its derivatives, especially low molecular weight heparin, through pulmonary route.

Another object of the present invention is to minimize the infection risk for health care personnel and uninfected people in the environment during the treatment of especially COVID-19, and viral lung diseases. The risk of infection to the environment is reduced by means of inhalation applications subject to the invention. The present invention enables the application such that the contamination of the room air is prevented by means of the closed system operation.

In the present invention, accumulation (condensation of drug/active substance-containing solutions) in the environment and in the upper respiratory tract is minimized, and thus, an aerosol with a low velocity that optimizes drug accumulation is produced by means of the administration of heparin or heparin derivatives via vibrating mesh nebulizers. Vibrating mesh technology nebulizers do not affect the stability of the drug/active substance since they do not generate heat.

In the present invention, drug localization in the lungs is much higher (20% and above) compared to other devices by means of the administration of heparin or heparin derivatives via a soft mist inhaler. The reason for this is that the droplet size range in a soft mist inhaler is so localized in the lungs that it is incomparable with a metered-dose inhaler (MDI), dry powder inhaler (DPI), jet, or ultrasonic nebulizer. In the soft mist inhaler, the user fits the device into his/her mouth via the mouthpiece and inhales through the mouth and subsequently, exhales through the nose, thereby minimizing the risk of exhalation through the mouth. Environmental contamination of saliva is prevented by means of creating a closed system. The soft mist inhaler used in the present invention has an application apparatus attached to the intubation tube that is developed for intubated patients, and this attachment makes the inhaler superior compared to present inhalers.

In the present invention, the pharmaceutical composition containing heparin or heparin derivatives may be arranged such that it is for single-use or reusable. A single-use dosage form is advantageous in the treatment of acute lung diseases since it does not carry the risk of contamination and does not require adding additional excipients (antioxidant, antimicrobial, etc.) to the formulation in order to provide stability. However, in the treatment of chronic diseases (COPD, asthma, etc.) the multi-dose form is more advantageous in long-term treatments when considering the patient compliance and cost since the patient uses the drug at home by himself/herself.

In the present invention administering heparin or heparin derivatives with a soft mist inhaler having a dosage-adjusting syringe enables that the dosage adjustment for the administration that targets the lung may be performed by the physician in the most sensitive way in response to the requirements of the patient. Said syringe system makes the implementation of patient-specific dosing by physicians significantly more practical in hospitals. In addition, the present heparin-containing syringes can be directly attached to the soft mist inhaler so that the treatment can be offered to the patients quickly in case it is required, thereby eliminating the supply problem. In addition, heparin and heparin derivatives can be pre-filled into the soft mist inhaler during the production process in the pharmaceutical factory in compliance with the single-use or multi-dose use and rendered ready to use by packaging.

### DESCRIPTION OF THE FIGURES

FIG. 1 illustrates the exploded view of the soft mist inhaler that is used for the administration subject to the invention.

FIG. 2 illustrates the schematic view of the passive vibrating mesh nebulizer that is used for the administration subject to the invention.

FIG. 3 illustrates the schematic view of the active vibrating mesh nebulizer that is used for the administration subject to the invention.

FIG. 4 illustrates the LMWH Lung Deposition Histogram

FIG. 5 illustrates the study Flow Chart (*3 patients have low compliance with the device therefore they were not given inhaled LMWH.)

FIG. 6 illustrates a view of the use of a soft mist inhaler, which is used for the administration subject to the invention, with respidrive.

FIG. 7 illustrates a view of the use of a soft mist inhaler, which is used for the administration subject to the invention, with respidrive.

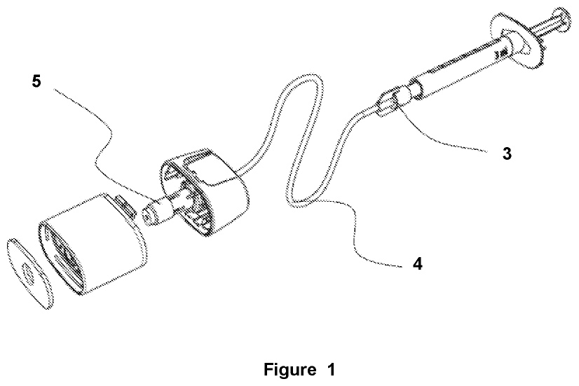

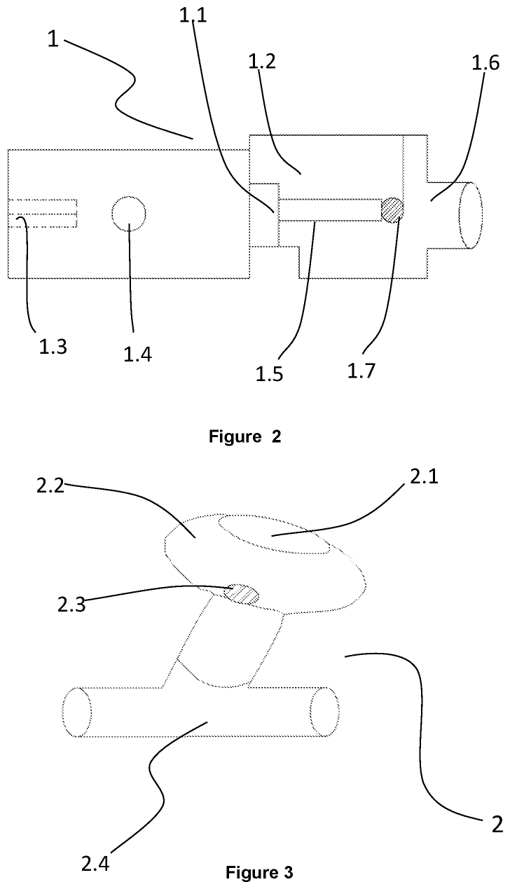

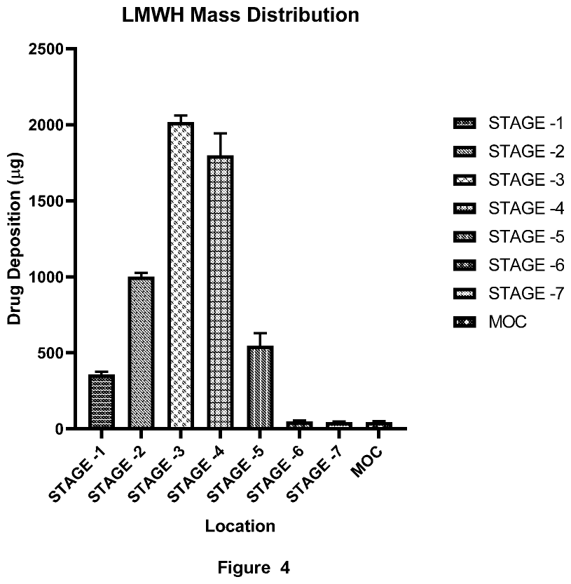

### DESCRIPTION OF ELEMENTS/PARTS/COMPONENTS OF THE INVENTION

The parts and components in the figures are enumerated for a better explanation of the present invention, and correspondence of every number is given below:
1—Passive Vibrating Mesh Nebulizer Device
   1.1—Piezoelectric Crystal
   1.2—Reservoir 1
   1.3—Batteries
   1.4—Operating Button
   1.5—Horn Converter
   1.6—Mouthpiece
   1.7—Mesh 1
2—Active Vibrating Mesh Nebulizer Device
   2.1—Cover
   2.2—Reservoir 2
   2.3—Mesh 2
   2.4—T-shaped Mouthpiece
3—Syringe/injector
4—Connection Tube
5—Soft Mist Inhalation Body
6—Respidrive

### DETAILED DESCRIPTION OF THE INVENTION

The present invention relates to the use of heparin or heparin derivatives, especially low molecular weight heparin (LMWH) for use in the treatment of especially COVID-19, viral lung diseases, acute and/or chronic lung diseases by means of soft mist inhaler or vibration mesh technology (VMT) nebulizer through inhalation route, and the pharmaceutical composition and dosage form pertaining to said use. The localization of the drug in the lungs (heparin composition therein) is 20% and above by means of the use of the pharmaceutical composition subject to the invention via soft mist inhaler or vibrating mesh technology (VMT) nebulizer through inhalation. In an embodiment of the present invention, the localization of the drug in the lungs (heparin composition therein) is 40%, 50%, or 60% by means of the use of a soft mist inhaler through inhalation. One of the reasons for selecting heparin in said treatments is that heparin is suitable for local administration to the lung. Heparin, in addition to anticoagulant features thereof, involves antiviral, anti-inflammatory, and mucolytic properties.

Heparin or heparin derivatives is an anticoagulant indicated in the treatment of acute lung injury caused by SARS-CoV-2 virus in the lungs, bronchial hypersensitivity due to inflammation, thromboembolism, histone release from damaged lung cells, and histone damage in the lungs, and further, in the treatment of hypoxemia, which is associated with acute respiratory distress syndrome (ARDS), and difficulty in breathing. In the present invention as heparin; low molecular weight heparin (LMWH), or unfractionated heparin (UFH), with their anticoagulant, anti-inflammatory, antiviral and mucolytic effects, can be used in the treatment of viral, acute, and/or chronic lung diseases. The heparin derivatives mentioned in the pharmaceutical composition subject to the invention can be all of the pharmaceutically acceptable derivatives of heparin. Heparin sodium salts, heparin esters, heparin ethers, heparin bases, heparin solvates, heparin hydrates, or their forms used as heparin prodrugs can be examples of heparin derivatives. All derivatives of LMWH and UFH, which are administered via inhalation in order to target the lungs, are suitable for being locally administered to lungs through the inhalation route by using a soft mist inhaler or a passive VMT nebulizer in the treatment of viral lung diseases, acute lung diseases and/or chronic lung diseases with COVID-19 being in the first place.

In the present invention, heparin or heparin derivatives can be added into a soft mist inhaler device or a vibrating mesh technology (VMT) nebulizer device at the production stage, or the solution that contains the active substance is packaged and stored in a dropper, prefilled syringe (PFS), ampoule, or vial, and said solution can be added into the device afterward, by patient or healthcare personnel before use in the hospital, or any environment.

In an embodiment of the present invention, an active or passive vibrating mesh technology (VMT) nebulizer is used as a vibrating mesh technology (VMT) nebulizer. Passive vibrating mesh nebulizer device (1) comprises; piezoelectric crystal (1.1), reservoir 1 (1.2), batteries (1.3), operating button (1.4), horn converter (1.5), mouthpiece (1.6), and mesh 1 (1.7). Active vibrating mesh nebulizer device (2), on the other hand, comprises; cover (2.1), reservoir 2 (2.2), mesh 2 (2.3), and t-shaped mouthpiece (2.4). The key component is a mesh plate (1.7), which contains a membrane perforated with precisely created holes. A piezo crystal (1.1) vibrates the mesh of aperture, which is acting as a micropump that draws fluid through the holes in order to create consistently sized fine particles with a diameter of 1-6 μm. The above-mentioned particle size is advantageous since particles with a diameter of 6-10 μm do not move beyond the larger lung airways. VMT Nebulizers produce a low-velocity aerosol that minimizes its accumulation (condensation of drug-containing solutions) in the environment and in the upper respiratory tract, thereby optimizing the drug accumulation. They do not generate heat, and therefore, they do not affect the stability of the drug.

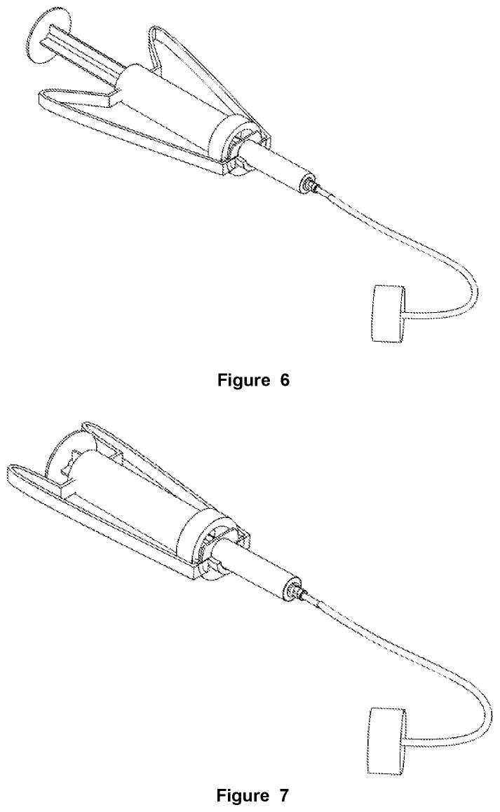

In an embodiment of the present invention heparin or heparin, derivatives are used by means of a soft mist inhaler through inhalation route in the treatment of especially COVID-19, viral lung diseases, acute lung diseases, and/or chronic lung diseases. In the present invention, the PulmoSpray® device available in the state of the art may be used as a soft mist inhaler. The soft mist inhaler comprises a soft mist inhalation body (5) including a special membrane therein, a connecting tube, a syringe, and optionally, in case the respidrive is in the prefilled form, a respidrive (6), or a similar holding system, in which the syringe will be placed (FIG. 6-7); and the soft mist inhalation body (5) provides maximum efficacy for the application. Here, what is meant by maximum efficacy is observing the balance between the highest active substance transfer and the lowest risk of infection. Aerosol droplets suitable for targeting the drug to the lungs are formed when the liquid passes through the membrane in the soft mist inhalation body (5) by means of pushing the liquid with pressure. Said soft mist inhaler is extremely suitable for safety in the use of COVID-19 treatment since it is fitted into the mouth of the patient as a closed system due to its mechanism, and the patient inhales the medicine from the device and exhales through the nose. The droplet size range of the soft mist inhaler, which is effective in the treatment of both COVID-19 and other viral lung diseases, is quite narrow due to the drug/active substance accumulation and ease of use provided by the soft mist inhaler in the lungs. The soft mist inhaler is fitted in the mouth with the mouthpiece, it is inhaled and exhaled through the nose; thus, closed-circuit respiration minimizes the environmental contamination of saliva. In addition, the soft mist inhaler has two more advantages that nebulizers do not have: dose accuracy and its practical use. In the soft mist inhaler, dose adjustment depending on weight and age may be performed easily by the physician in the hospital, with patient-specific flexibility by means of the syringe system attached to the device. The parenteral forms of heparin, which are commercially available as a prefilled syringe may also be used immediately in the patients via use method subject to the invention without requiring an additional formulation step due to the fact that the device works with a syringe system.

In the present invention, there is a syringe (injector) with dosing function in the soft mist inhaler used for the administration of heparin or its derivatives in the treatment of especially COVID-19, viral lung diseases, acute lung diseases, and/or chronic lung diseases. The dosage adjustment for the application that targets the lung may be performed by the physician in the most sensitive way in response to the requirements of the patient by means of said special syringe. Said syringe system makes the implementation of patient-specific dosing by physicians significantly more practical in hospitals. In addition, the parenteral dosage form of heparin and heparin derivatives, which is commercially available as a ready-to-use syringe may be directly connected to the soft mist inhaler used in the present invention. The fact that the parenteral form of heparin and heparin derivatives is directly compatible with the device enables the “formulation-device-administration” triangle to operate in the most efficient way, and the fastest application to the patients, especially to the elderly in the risk group (>65 years) in these pandemic conditions competing with time. Heparin or heparin derivatives pass through the inter-device connection tube (4) after the syringe (3), and heparin or its derivatives become the aerosol droplets in the particle size range that may be localized in the lungs, and thus, it can be administered to the lungs via soft mist inhaler by means of the nozzle mechanism in the soft mist inhalation body (5). The soft mist inhaler works with an active mechanism that does not require propellant; the energy required for aerosol production is provided from the inhaler itself, and thus, it is independent of the respiratory capacity of the patient. The size range of the aerosol droplets released from the device is in the range of 2-6 micrometers and said aerosol droplets target the lungs. Therefore, the present invention allows for an efficient treatment. Another advantage of the soft mist inhaler is that dosing is performed by means of a syringe.

In a preferred embodiment of the present invention, low molecular weight heparin (LMWH) is used in order to be administered by means of soft mist inhaler or vibrating mesh technology (VMT) nebulizer for the treatment of especially COVID-19, viral lung diseases, acute lung diseases, and/or chronic lung diseases. Low molecular weight heparin (LMWH), which is a member of the anticoagulant drug group, displays high efficacy by means of providing local involvement in the lungs when inhaled through the mouth. LMWH, due to its antiviral, anti-inflammatory, and mucolytic properties is also effectively used in the treatment of COVID-19 and other viral lung diseases.

In the present invention, the pharmaceutical composition administered through the inhalation route contains heparin or heparin derivative, and a carrier solution that displays heparin solvent properties. The pharmaceutical composition that is disclosed in the present invention and that contains heparin or heparin derivative therein may also be referred to hereinafter as heparin composition or heparin solution. The heparin-containing composition to be inhaled contains 4000-25000 IU of heparin or heparin derivative that is dissolved in carrier solution (preferably water for injection), preferably low molecular weight heparin (LMWH). The solvent may be aqueous or non-aqueous within heparin composition. A dosage form may be formulated with one or a mixture of more than one pharmaceutically acceptable solvent and can be, but not limited to, glycerol, propylene glycol, polyethylene glycol, polypropylene glycol, ethyl alcohol, isopropyl alcohol, water, mineral oil, peanut oil, and corn oil. The pharmaceutical solvents may be used to prepare the formulation concentrate as well as used for reconstitution of the dosage form. Pharmaceutically acceptable solvents such as water, ethyl alcohol, isopropyl alcohol are evaporable and are usually used to dissolve or disperse the medicament and excipients in the formulation concentrate. Glycerol, propylene glycol, and polyethylene glycol are co-solvents and are used to assist in the solubilization of water-insoluble or poorly water-soluble medicaments in the formulation concentrate. Pharmaceutically acceptable reconstituting solvents such as sterile water for injection, water for inhalation, sterile normal saline solution (0.9% NaCl), sterile half saline solution (0.45% NaCl), sterile phosphate buffer solution (pH 4.5-7.4), and/or sterile 5% dextrose solution are used for reconstitution of the dosage form to form a solution or a fine particle suspension of pharmaceutically active substance prior to oral or nasal inhalation via VMT nebulizer or soft mist inhaler.

The composition subject to the invention comprises 4000 IU, 6000 IU, 8000 IU, or 10000 IU heparin or heparin derivative. More specifically, the composition subject to the invention may be a sterile inhaled solution comprising of 4000 IU/ml, 6000 IU/ml, 8000 IU/ml, or 10000 IU/ml of heparin, especially LMWH or UFH, which is contained in an injectable water or water for inhalation or physiological saline or half physiological saline or phosphate buffer.

In a preferred embodiment of the present invention, the composition is a sterile inhaled solution in the 4000 IU/mL concentration that is obtained by dissolving 4000 IU of LMWH in 1 mL of carrier solution. The carrier solution in the composition is used up to the required milliliter (ml) in order to obtain heparin solution at a concentration of 4000 IU/ml; wherein the carrier solution acts as both carrier and solvent, and is selected among the water for injection, water for inhalation, physiological saline (0.9% NaCl), or half physiological saline (0.45% NaCl), or phosphate buffer (pH 7.4). Heparin solution at a concentration of 4000 IU/ml is packaged and used as a one-time administration dose. However, in case it is desired to be used in pediatric patient groups, the dose adjustment of the user is performed over said one-time dose. The only administration route of the final composition is through inhalation, however, targeting of local or systemic effect may vary according to the disease that desired to be treated.

Heparin composition, in addition to heparin or heparin derivative, may contain at least a different active substance or at least one excipient. The heparin referred to here is preferably LMWH, or UFH, or any derivative thereof. In an embodiment of the present invention, the active substances that may be used in addition to heparin or heparin derivatives are given in three pharmacological groups, and any combination of these may be used together;
1—Those with the purpose of mucolytic effect: Mannitol, acetyl cysteine, or hypertonic (3-20% NaCl, w/v) physiological saline.
2—Those with the purpose of eliminating the oxidative stress in the lungs: Ascorbic acid and its derivatives
3—Corticosteroids with the anti-inflammatory properties: Budesonide, beclomethasone dipropionate, fluticasone, mometasone, or dexamethasone.

In an embodiment of the present invention, mannitol or acetyl cysteine may be added to the low molecular weight heparin (LMWH) or unfractionated heparin (UFH) solution in the pharmaceutical composition containing heparin or heparin derivative. Thus, also the opening effect of the mucus plug in the lungs is provided. In another embodiment of the present invention, heparin solution, in which mannitol is added into LMWH or UFH heparin solution, is prepared hypertonic (3-20% NaCl, w/v), thereby, providing the opening effect of the mucus plug.

In the heparin composition, excipient(s) may be used in case a different active substance is used in addition to heparin or heparin derivatives, or directly in addition to the heparin composition. The pharmaceutical composition can contain at least one excipient selected from tonicity adjusting excipients, pH adjusting or buffering agents, tonicity adjusting agents, antioxidants, antimicrobial preservatives, surfactants, solubility enhancers (co-solvents), stabilizing agents, excipients for sustained release or prolonged local retention, wetting agents, dispensing agents, taste-masking agents, sweeteners, and/or flavors. These excipients are used to obtain an optimal pH, viscosity, surface tension, and taste, which support the formulation stability, aerosolization, tolerability, and/or the efficacy of the formulation upon inhalation.

One or more co-solvents (solubility enhancer) may be included in the heparin pharmaceutical composition to aid the solubility of the active substance and/or other excipients. Examples of pharmaceutically acceptable co-solvents include, but are not limited to, propylene glycol, dipropylene glycol, ethylene glycol, glycerol, ethanol, polyethylene glycols (for example PEG300 or PEG400), methanol, polyethylene glycol castor oil, polyoxyethylene castor oil, and/or lecithin.

Stabilizing agents which can be used for the heparin composition are antioxidant and chelating agents that are capable of inhibiting oxidation reaction and chelating metals, respectively, to improve stability of pharmaceutically active substance and excipients. Dosage forms may be formulated with one or more pharmaceutically acceptable stabilizing agents at a concentration suitable for the intended pharmaceutical applications, and may be, but not limited to, chelating agents such as disodium edetate (Ethylenediaminetetraacetic acid, EDTA) or its sodium salt, citric acid, sodium citrate, vitamin E, ascorbic acid, ascorbyl palmitate, butylated hydroxyanisole, butylated hydroxytoluene, hypophosphorous acid, monothioglycerol, propyl gallate, sodium bisulfite, sodium metabisulfite, sodium formaldehyde sulfoxylate, thiourea, lysine, tryptophan, phenylpropyl glycine, glycine, glutamic acid, leucine, isoleucine, serine, tea polyphenols, ascorbyl palmitate, hydroxymethyl ester, hydroxyethyl tetramethyl piperidinol, bis (2, 2,6,6-tetramethyl-4-piperidyl) sebacate, polysuccinate (4-hydroxy-2,2,6,6-tetramethyl-1-piperidinylethanol) ester, 2-[2-hydroxy-4-[3-(2-ethylhexyloxy)-2-hydroxypropoxy]phenyl]-4,6-bis (2,4-dimethylphenyl) and/or 1,3,5-triazine.

Antioxidants, which are natural or synthetic substances that prevent or interrupt the oxidation of active agents and/or oxidative injury in stressed tissues and cells, can be used in the heparin composition. Antioxidants that can be used in the heparin composition can be adjuvants that are oxidizable themselves (i.e. primary antioxidants) or adjuvants that act as reducing agents (i.e. reducing antioxidants), such as tocopherol acetate, lycopene, reduced glutathione, catalase, and/or peroxide dismutase. Other adjuvants used to prevent oxidative reactions are synergistic antioxidants, which do not directly act in oxidation processes, but indirectly via the complexation of metal ions that are known to catalyze oxidation reactions. Frequently used synergistic antioxidants are ethylenediamine tetraacetic acid (EDTA) and its derivatives. Further useful antioxidants (primary, reducing, and/or synergistic anti-oxidizing working mechanism) are ascorbic acid and/or its salts, esters of ascorbic acid, fumaric acid and/or its salts, malic acid and/or its salts, citric acid and/or its salts, butyl hydroxy anisole, butyl hydroxy toluene, propyl gallate and/or maltol. As an alternative to generally used antioxidants, substances such as acetylcysteine, R-cysteine, vitamin E TPGS, pyruvic acid and/or its magnesium and/or sodium salts, gluconic acid and/or its magnesium and/or sodium salts, might also be useful in formulations for inhalation. The salts of gluconic acid have the additional advantage that they have been described to have an anti-oxidizing effect on stressed tissues and cells, which can be particularly advantageous in the treatment of inflammations, as oxygen radicals induce and perpetuate inflammatory processes. Also, pyruvate salts are believed to have such in vivo anti-oxidizing effects. An additional measure to prevent oxidation and to contribute to the prevention of the undesired discoloration is the replacement of oxygen above the solution by an inert gas but not limited to such as nitrogen or argon.

Antimicrobial preservatives can be used in the heparin composition to inhibit the growth of microorganisms. Dosage forms may be formulated with one or more pharmaceutically acceptable antimicrobial preservatives at suitable concentrations to prevent microbial growth. Compositions for administration to the lungs or nose may contain one or more excipients, may be protected from microbial or fungal contamination or growth by the inclusion of one or more preservatives. Examples of pharmaceutically acceptable antimicrobial agents or preservatives include, but are not limited to, quaternary ammonium compounds (e.g., benzalkonium chloride, benzethonium chloride, cetrimide, cetylpyridinium chloride, lauralconium chloride and/or myristyl picolinium mercuric chloride), thimerosal alcoholic agents (e.g. chlorobutanol, phenylethyl alcohol and/or benzyl alcohol), antibacterial esters (e.g. parahydroxybenzoic acid esters), chelating agents such as disodium edetate (EDTA) other antimicrobial agents such as chlorhexidine, chlorocresol, sorbic acid and/or its salts (such as potassium sorbate) and polymyxin. Examples of pharmaceutically acceptable antifungal agents or preservatives include, but are not limited to, sodium benzoate, sorbic acid, sodium propionate, methylparaben, ethylparaben, propylparaben, butylparaben, ethyl p-hydroxybenzoate, and/or n-propyl p-hydroxybenzoate.

pH adjusting or buffering agents can be used in the heparin composition to adjust or maintain the pH of the pharmaceutical dosage form to the desired range for the following reasons: to provide an environment for better product stability that pharmaceutical active substance may express better chemical stability within a certain pH range, or to provide better comfort for the patient at administration. Extreme pH may create irritation and/or discomfort to the site of administration, and provide a pH range for better antimicrobial preservative activity. The heparin composition can comprise one or more excipients to adjust and/or buffer the pH value of the solution. For adjusting and optionally buffering pH, physiologically acceptable acids, bases, salts, and/or combinations thereof may be used. Excipients often used for lowering the pH value or for application as an acidic component in a buffer system are strong mineral acids, in particular sulfuric acid and hydrochloric acid. Also, inorganic and organic acids of medium strength, as well as acidic salts, may be used such as phosphoric acid, citric acid, tartaric acid, succinic acid, fumaric acid, methionine, acidic hydrogen phosphates with sodium or potassium, lactic acid, and/or glucuronic acid. Excipients suitable for raising the pH or as a basic component in a buffer system are, in particular, mineral bases such as sodium hydroxide or other alkaline earth hydroxides and oxides such as magnesium hydroxide and calcium hydroxide, ammonium hydroxide, and basic ammonium salts such as ammonium acetate, as well as basic amino acids such as lysine, carbonates such as sodium or magnesium carbonate, sodium hydrogen carbonate, and citrates such as sodium citrate. The heparin composition can comprise a buffer system consisting of two components. One of the most preferred buffer systems contains citric acid-sodium citrate, citric acid-phosphoric acid disodium hydrogen, potassium dihydrogen phosphate-disodium hydrogen phosphate, or citric acid-sodium hydroxide, trometamol, disodium phosphate (for example dodecahydrate, heptahydrate, dihydrate, and anhydrous forms thereof) and/or sodium mixtures. Nevertheless, other buffering systems may also be used.

A tonicity adjusting agent is one or more pharmaceutical excipients that are osmotically active, and which are used in common practice for the purpose of adjusting the osmolality or tonicity of liquid pharmaceutical formulations. Mainly tonicity adjusting agents are used to enhance the overall comfort to the patient upon administration. A tonicity adjusting agent can be used in the heparin composition selected from sodium chloride, mannitol, or dextrose. Other salts that can be used in the heparin composition for adjusting tonicity are sodium gluconate, sodium pyruvate, and/or potassium chloride. Also, carbohydrates can be used for this purpose. Examples are sugars such as glucose, lactose, sucrose, or trehalose, sugar alcohols such as xylitol, sorbitol, and/or isomaltol. Alternately, the dosage form may be formulated without the addition of a major tonicity adjusting agent. The desired tonicity of the dosage form is achieved by reconstituting with a sterile isotonic saline solution.

The surface tension of a liquid composition is important for optimal inhalation. Compositions with a desirable surface tension are expected to show a good spreadability on the mucous membranes of the respiratory tract. In order to enable the formulation to be atomized smoothly and form uniform and stable aerosol particles to be absorbed by the patient, optimal surface tension is needed. Furthermore, the surface tension might need to be adjusted to allow a good emptying of the composition from its primary package. Surfactants are materials with at least one relatively hydrophilic and at least one relatively lipophilic molecular region that accumulates at hydrophilic-lipophilic phase interfaces and reduces the surface tension. The surface-active materials can be ionic or non-ionic. Particularly preferred surfactants are those that have good physiological compatibility and that are considered safe for oral or nasal inhalation. A preferred surfactant in the heparin composition can be tyloxapol, polysorbates, polysorbate 20, polysorbate 60, polysorbate 80, lecithin, vitamin E TPGS, macrogol hydroxystearates, and/or macrogol-15-hydroxystearate. The surfactant used in the heparin composition might also comprise a mixture of two or more surfactants, such as polysorbate 80 in combination with vitamin E TPGS.

In some of the embodiments of the invention, also taste-masking agents or sweetening agents, or flavoring agents, can be used as an excipient. A bad taste of formulations for inhalation is extremely unpleasant and irritating. The bad taste sensation upon inhalation results from direct deposition of aerosol droplets in the oral and pharyngeal region upon oral inhalation, from the transport of drug from the nose to the mouth upon nasal inhalation, and from the transport of the drug from the respiratory tract to the mouth related to the mucociliary clearance in the respiratory system. A taste-masking agent is any pharmaceutically acceptable compound or a mixture of compounds capable of improving the taste of an aqueous system, regardless of the mechanism by which the improvement is brought about. For example, the taste-masking agent may cover the poor taste, i.e. reduce the intensity by which it is perceived, or it may correct the taste by adding another, typically more pleasant, flavor to the composition, thereby improving the total organoleptic impression. Other taste-masking mechanisms are complexation, encapsulation, embedding, or any other interaction between drugs and other compounds of the composition. A taste-masking agent which can be used in the heparin composition is selected from the group of pharmaceutically acceptable sweeteners such as saccharin, aspartame, cyclamate, sucralose, acesulfame, neotame, thaumatin, and/or neohesperidine, including salts and solvates thereof such as the sodium salt of saccharin and the potassium salt of acesulfame. Furthermore, sugars such as sucrose, trehalose, fructose, and lactose, or sugar alcohols such as xylitol, mannitol, or isomalt can be used. Further useful taste-masking agents include pharmaceutically acceptable surfactants, alkaline earth metal salts, organic acids such as citric acid and lactic acid, and/or amino acids such as arginine. Also, aromatic flavors, such as the ingredients of essential oils (menthol, thymol, or cineol) may be used in the heparin composition to improve the taste and tolerability of the composition according to the invention.

Wetting or dispensing agents can be used in the heparin composition to increase wettability and assist in dispersing water-insoluble or poorly water-soluble particles. For water-insoluble and poorly water-soluble medicaments, the addition of one or more wetting or dispersing agents to the dosage formulation can help the release of the impregnated pharmaceutical active substance particles from the supporting material into the reconstituted solution and can help the dispersion of the particles to form a fine suspension. Examples of pharmaceutically acceptable wetting and dispersing agents suitable for oral or nasal inhalation for the heparin composition are poloxamers, oleic acid or its salts, lecithin, hydrogenated lecithin, sorbitan fatty acid esters, oleyl alcohol, phospholipids including but not limited to phosphatidylglycerol, phosphatidylcholine, polyoxyethylene fatty alcohol ethers, polyoxypropylene fatty alcohol ether, polyoxyethylene fatty acid ester, glycerol fatty acid esters, glycolipid such as sphingolipid and sphingomyelin, polyoxyethylene glycol fatty acid ester, polyol fatty acid esters, polyethylene glycol glycerol fatty acid esters, polypropylene glycol fatty acid esters, ethoxylated lanolin derivatives, polyoxyethylene fatty alcohol, polyoxyethylene sorbitan fatty acid esters, polyoxyethylene stearate, propylene glycol alginate, dilauryldimethylammonium chloride, D-a-tocopheryl-PEG 1000 succinate, Polyoxy 40 stearate, polyoxyethylene-polyoxypropylene block copolymers, polyoxyethylene vegetable oils, fatty acid derivatives of amino acids, glyceride derivatives of amino acids, benzalkonium chloride and/or bile acids.

In the present invention, the primary packaging to be used for LMWH should be transparent, or amber-colored, or opaque, and it is made of a pharmaceutical-grade material that is biologically compatible with the content of the heparin composition. The material of the chamber that will contain the heparin composition may be glass or synthetic material. The formulation may be packaged in a single dose or multi-dose form. The formulation may be pre-filled to the inhaler or may be in a form that allows the formulation to be provided to the inhaler during use. Unit-dose respiratory drugs are packaged in soft plastic containers, which are generally formed of low-density polyethylene (LDPE) or LPDE in order to control costs and facilitate the opening of containers. In the present invention, the primary packaging to be used for LMWH may be made of glass material.

Said composition may be single-use or reusable. In case said composition is reusable, it may also contain antioxidant agent, antimicrobial preservative, vitamin, pH adjusting agent, buffering agent, surfactant, tonicity adjusting agent, stabilizer, complexing agent. In case it is single-use, only carrier solution (water for injection, inhalation water or phosphate buffer, etc.) will be sufficient as an excipient. However, an additional excipient is also used in the case of adding a different active substance to the single-use composition. In case it is reusable or in combination with other active substances, substances from the excipient groups that are indicated in detail above may be added to the formulation content.

The composition subject to the invention is prepared in solution form, and it is administered to the patient through inhalation route by means of soft mist inhaler or VMT nebulizer devices. The heparin composition may be a solution, suspension, or emulsion containing heparin or a heparin derivative. The composition subject to the invention, or in addition to the heparin composition, the composition in combination with antivirals, mucolytic agents, vitamins, or corticosteroids are applied in the treatment of viral or acute or chronic lung diseases; especially COVID-19, influenza, tuberculosis, cystic fibrosis, chronic obstructive pulmonary disease (COPD), asthma, bronchitis, acute respiratory distress syndrome (ARDS), hypoxemia, pulmonary embolism, pulmonary hypertension, acute lung injury (ALI) and/or burn associated with ALI. The patient groups to which the composition subject to the invention may be applied are inpatients, outpatients, or home care patients.

Indications, for which the heparin in a sterile solution dosage form to be administered via a soft mist inhaler may be used in lungs are grouped under three main titles within the scope of the present invention:
1—Viral lung diseases: COVID-19, influenza
2—Acute lung diseases: Acute pulmonary infection, bronchitis, acute lung injury (ALI), burn associated with ALI, acute pulmonary embolism, bronchial hyperresponsiveness, Acute Respiratory Distress Syndrome (ARDS), hypoxemia.
3—Chronic lung diseases: Pulmonary embolism, pulmonary hypertension, cystic fibrosis, idiopathic pulmonary fibrosis, asthma (exercise-induced asthma, mild asthma, cold-induced asthma, etc.), sarcoidosis, chronic pulmonary embolism, COPD.

Impactors were used based on the method in European Pharmacopoeia 2.9.18 (EP Monograph 2.9.18, 2010) in order to simulate the distribution of sterile inhaled formulations, which are obtained within the scope of the present invention, in the lungs. The device is connected to the soft mist inhaler. Aerodynamic particle size data are interpreted as mean mass aerodynamic diameter (MMAD), geometric standard deviation (GSD), and fine particle fraction (percentage of particles with aerodynamic particle size less than 5 μm) values The dispersed phase of the aerosol prepared from the compositions of the invention exhibits a mass median aerodynamic diameter (MMAD) preferably from about 1 to about 6 μm and more preferably from about 2 to about 4.5 μm or from about 1.5 to about 4 μm. Aerodynamic particle size is very important in drug delivery to the lungs. In local delivery to the lungs, particles in the range of 1-6 μm are targeted to the bronchi and bronchioles. The LMWH solution aerosol localization studies showed that the mean MMAD value was between 1-6 μm and the mean FPF value was between 10%-60% and more preferably 5.3 μm and 44%, respectively.

Inhalation of LMWH with a nebulizer (UFH) was shown to be highly effective for acute lung injury and acute respiratory damage in previous studies. Therefore, patients with worse clinical courses have been given priority (by ethical choice) for the treatment of the inhaled LMWH. LMWH was applied to the Study Group with a dose of 4000 IU twice a day, in addition to Subcutaneous Low Molecular Weight Heparin. The control group received only the standard therapy.

Patients were eligible to receive inhaled LMWH if they were male or non-pregnant female aged 18 years or above, had a positive reserve transcriptase-polymerase chain reaction (RT-PCR) test of the nasopharyngeal swab for COVID-19 and pneumonia confirmed by a Computed Tomography (CT), or had a negative reserve transcriptase-polymerase chain reaction (RT-PCR) test of the nasopharyngeal swab for COVID-19 but are clinically, radiologically, and biochemically suggestive of the diagnosis of COVID-19. Any other possible diagnoses were excluded. Exclusion criteria were patients not willing to give informed consent, pregnancy, and allergy to heparin. A full list of inclusion and exclusion criteria can be found in Table 1.

TABLE 1
Study inclusion and exclusion criteria
Study Eligibility Criteria

| | |
|---|---|
| Inclusion | Written informed consent under no pressure |
| Criteria | Positive reserve transcriptase-polymerase chain reaction (RT- |
| | PCR) test of the nasopharyngeal swab for COVID-19, and |
| | pneumonia confirmed by a Computed Tomography (CT) |
| | Negative reserve transcriptase-polymerase chain reaction |
| | (RT-PCR) test of the nasopharyngeal swab for COVID-19, but |
| | radiological and biochemical examination unambiguously |
| | suggest COVID-19, when other possible diagnoses were |
| | excluded. |
| | Patients aged ≥18 years |
| Exclusion | Patients with pregnancy |
| Criteria | History of allergy to heparin and associated drugs |
| | Patients not willing to give an informed consent |

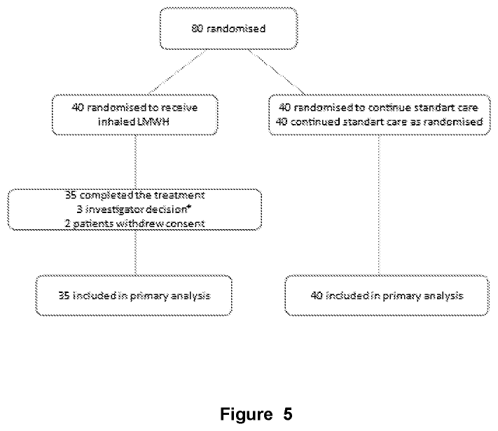

Informed written consent was obtained from patients prior to the enrolment. For those patients, who were not able to give informed written consent, it was obtained from the patient's first-degree relatives upon a briefing about the study. Patients who were not willing to give a written informed consent were not included in the enrolment. Additional information about the study design is available in the study flow chart (FIG. 5).

The primary outcome of the study was to evaluate oxygen saturation, fever, and other vital signs during the routine follow-up of the patients. In addition to these, changes in the biochemical parameters such as C-reactive protein, ferritin, D-dimer, Neutrophil count, Lymphocyte count, and the ratio of Neutrophil to Lymphocyte were evaluated. The secondary outcome of the study was the evaluation of rationality for oxygen therapy, and whether there was a need for intubation and intensive care unit treatment for these patients.

The study for the present invention consists of two groups: Device and Control groups. The Device Group entails 35 COVID-19 patients (20M/15F), while the Control Group entails 40 patients (25M/15F) (see Table 1). The Device Group was treated with a novel device and an accompanying novel formula, whereas the Control Group was given the standard COVID-19 treatment. The average age of the Device Group is 60.01±10.04 and of the Control Group was 59.62±14.60 (see Table 2).

TABLE 2
Baseline patient characteristics

| | Soft mist inhaler | |
|---|---|---|
| | heparin group | Control group |
| Demographics | | |
| Age (y) | 60.02 ± 10.04, n = 35 | 59.62 ± 14.60 n = 40 |
| Female | n = 15 (43%) | n =15 (37.5%) |
| Male | n = 20 (57%) | n = 25 (62.5%) |
| Body mass index (kg/m²) | 1.92 ± 0.21, n = 35 | 1.97 ± n = 40 |
| Co-morbidities | | |
| Tobacco smoking | n = 3 (8.5%) | n = 6 (15%) |
| COPD | n = 2 (5.7%) | n = 2 (5%) |
| Cardiac Disease | n = 7 (20%) | n = 9 (22.5%) |
| Diabetes Mellitus | n = 8 (22.8%) | n = 10 (25%) |
| COVID-19 duration | | |
| Time from symptom | 3.54 ± SD, n = 35 | 4.4 ± 4.18, n = 40 |
| onset (d) | | |

According to the standard COVID-19 treatment algorithm used in the experiments for this invention, patients were given Favipravir 200 mg 16 tablets the first day, and 200 mg 6 tablets per day for the following 4 days, also subcutaneous LMWH and methylprednisolone are given to patients due to the clinical condition. Both control and device groups were given subcutaneous LMWH and intravenous to methylprednisolone 40 mg/day. Following the hospital admission of patients, Computerized Tomography of lungs was taken with low dose radiation. Parenchymal findings were categorized into severity degrees according to the following criteria: lobe involvement, involved area of lobe, patch, or diffuse as shown in Table 3. The average radiological severity score of patients in the Device Group was 5.6±1.5, in the Control Group average score was 6.4±1.8. There is no significant difference in radiological severity between the device and the control groups.

TABLE 3
Radiological severity index

| Degree of | |
|---|---|
| severity | Explanation |
| 1 | One lobe less than 25% of lobe area |
| 2 | One lobe more than 25% of lobe area |
| 3 | Unilateral and more than one lobe less than 25% of each |
| | lobe area |
| 4 | Unilateral and more than one lobe less than 25% of each |
| | lobe area |
| 5 | Bilateral patch lesions all lobes |
| 6 | Bilateral but the whole of one but not all lobes |
| 7 | Bilateral, all lobes, diffuse but less than 25% of each lobe |
| | area |
| 8 | Bilateral, all lobes, diffuse, and 25-50% each lobe area |
| 9 | Bilateral, all lobes, diffuse, and 50-75% each lobe area |
| 10 | Bilateral, all lobes, diffuse, and more than 75% of each lobe |
| | area |

The patients of both groups present with primarily respiratory distress as typical of COVID-19, including incessant coughing, sputum production, and shortness of breath, and other symptoms such as high fever and extreme fatigue as shown in Table 4. The fever data of patients at the beginning of treatment, clinical parameters of the peripheral oxygen saturation along with CRP, Ferritin, Leukocyte count, Neutrophil/Lymphocyte ratio, and other laboratory parameters are shown in Tables 4.

Clinically speaking, shortness of breath and sputum production were significantly higher in the Device Group (<0.01). Coughing was not significantly different within, and in comparison, of, both groups. In terms of clinical symptom scoring, the Device Group had a significantly higher symptom score, meaning that (statistically on the average) members of this group experienced COVID-19 with much more severe symptoms. Inhaled LMWH had been shown to be effective in improving lung injury in previous studies (citation?). For this reason, patients with more severe symptoms were given the priority (by medically induced ethical choice) to receive inhaled LMWH (Table 4).

TABLE 4
Patient parameters in Device and Control groups

| | | Device | | Control | |
|---|---|---|---|---|---|
| Distribution | Cough | 25 | (%71.4) | 27 | (%67.5) |
| of | Mucus | 10 | (%28.5) | 1 | (%2.5) |
| Symptoms | Dyspnea | 32 | (%91.4) | 23 | (%57.5) |
| (number of | | | | | |
| cases) | | | | | |
| State of | Hypoxemic | 33 | (%94.3) | 11 | (%27.5) |
| Hypoxemia | Normoxemic | 2 | (%5.7) | 29 | (%72.5) |

| | | | |
|---|---|---|---|
| Room air | | | |
| Clinical | Fewer | 36.6 ± 0.4 | 37.4 ± 0.8 |
| Parameters | Sp0₂ (with 0₂ | 95 ± 2.5 | 93.8 ± 2.89 |
| | supplementation) | | |
| Laboratory | CRP median | 41 | 72 |
| Parameters | CRP min | 1 | 2 |
| | CRP max | 232 | 372 |
| | Ferritin median | 698 | 487 |
| | Ferritin min | 102 | 23 |
| | Ferritin max | 3713 | 5785 |
| | Leukocyte median | 8400 | 5675 |
| | Leukocyte min | 3000 | 2250 |
| | Leukocyte max | 45200 | 13610 |
| | Neutrophil/Lymphocyte | 11.28 | 5.22 |
| | median | | |
| | Neutrophil/Lymphocyte | 1.45 | 0.97 |
| | min | | |
| | Neutrophil/Lymphocyte | 27.66 | 20.86 |
| | max | | |

The average of fever (body temperature measured in Celsius) at the admittance in the Control Group patients was higher than that of the Device Group (<0.001), while there was no significant difference between the Day 1 oxygen saturation values. The marked difference in the fever is an indication that Control Group patients have suffered from a more intense form of COVID-19 (Table 4). This is a crucial feature in the study related to the present invention, namely by the admittance some specific parameters fared worse in the Control Group, and these parameters had not improved with the standard therapy, which arguably implies that therapy of the present invention could have been much more effective.

The Peripheral Saturation value of 95% and more at the beginning of the treatment was predetermined as “normal” for both device and control groups, and any value below was determined as hypoxemia. Accordingly, in the Device Group, only 2/35 (%5.7) cases were normoxemic, and 33/35 (%94.3) cases were hypoxemic. In the Control Group, in sharp contrast, 29/40 (%72.5) cases were normoxemic, and 11/40 (%27.5) were hypoxemic, which suggests that the Device Group as of Day 1 of the treatment had a greater hypoxemic lead and more critical patients (Table 4).

Of the laboratory parameters, CRP was significantly higher (<0.01) in the Control Group, while Ferritin, Leukocyte, Neutrophil/Lymphocyte ratios were significantly higher (<0.01) in the Device Group. The upper limits of the D-Dimer value were found not to be significantly different between the two groups (Mann-Whitney U). The Device Group included more severe patients compared to the Control Group based on laboratory parameters.

The severity of hypoxemia and the peripheral oxygen saturation of patients were measured on the 1st and 10th (last study day) days of the treatment, based on the patients' response to the device of oxygen therapy given. Each therapy implies a different level of severity. The threshold value was determined as 95% and above.

The severity levels were categorized as follows: Level 1, if the peripheral oxygen saturation improved with an oxygen therapy up to 6 Lt/min via a nasal cannula; Level 2, if it can be improved with a 500m1 reservoir oxygen mask with 15 Lt/min oxygen treatment; Level 3, if it can be improved with high flow oxygen therapy; Level 4, if intubation was the only choice (Table 5). A marked difference exists between the Device and Control group in terms of the number of patients in the room air category, by the end of the treatment of 10 days. The Device Group is mainly composed of severe patients, whereas 40 percent of the Control Group is non-severe. This difference provides a clear picture of how patients may fare with the existing methods of oxygen supply as opposed to the device supply proposed in this study.

TABLE 5
Oxygen therapy method for Device Group and Control
Group on the 1^{st} and 10^{th} day of treatment

| | Treatment day 1 | | Treatment day 10 | |
|---|---|---|---|---|
| Oxygen supply | Device | Control | Device | Control |
| method | n(%) | n(%) | n(%) | n(%) |
| 0: Room air | 0(5.7%) | 16(40%) | 27(77.1%) | 29(72.5%) |
| 1: Nasal cannula | 13(39.5%) | 15(37.5%) | 5(14.3%) | 6(15%) |
| 2: Reservoir oxygen | 12(31.6%) | 7(17.5%) | 2(5.7%) | 1(2.5%) |
| mask | | | | |
| 3: High Flow oxygen | 8(23.7%) | 2(5%) | 1(2.9%) | 1(2.5%) |
| 4: Intubation | 0(0%) | 2(0%) | 0(0%) | 3(7.5%) |

Patients in the Device Group required a highly significant (p<0.01) intensive oxygen therapy to overcome hypoxemia. At the end of the 10-day treatment period, the improvement of patients' hypoxemia as induced by the method of oxygen supply is shown in Table 5 (Device Group and Control Groups).

In the Device Group, 13/13 patients with hypoxemia, who were supplied oxygen via nasal cannula, were normoxemic by the end of the treatment. Of the Device Group, 16/35 cases (45.7%) had improved 1 stage, 12/35 cases (34.3%) 2 stages, and 3/35 cases (8.6%) 3 stages for the better clinical outcome. At the end of the treatment, there were no cases of intubation as the majority had achieved the state of “room-air-supply”.

In the Control Group, however, the 10-day period recorded a more heterogeneous outcome. For instance, of the nasal cannula group as of Day 1, 4/15 cases (26.6%) had no change in their status. However, 3 patients had to be intubated at some point within the 10-day period. In terms of overall improvement rate, 14/40 cases (35%) improved one severity level, 2/40 cases (5%) improved 2 severity levels, and only 1/40 cases (2.5%) improved 3 severity levels. This was 3 patients in the Device Group. The greatest contrast is in the improvement of 2 levels, in the Control Group, only 5 percent could be healed 2 severity levels by the standard therapy. In particular, however, the fact that 3 (7.5%) cases with nasal cannula have slipped into intubation severity implies that the outcomes of current treatments may be quite divergent in terms of patient response. Even if many may be healed by standard methods, some patients do indeed slip into “more severe” levels, which includes intubation. Also, in the Device Group, improvements were more of “wider shifts” across the levels such as improving more than one level, meaning more patients benefited from the device, as the weight of cases had shifted to the less severity levels more homogeneously.

The reduction in the amount of oxygen supply given to patients in the Device Group is significant in comparison to the Control Group. This difference was clearly pronounced in the subgroup receiving reservoir oxygen mask or high flow oxygen therapy. Moreover, in the Device Group, there was no case of intubation, whereas in the Control Group 3 patients had to be intubated, indicating that the probability of risk of intubation is markedly reduced for the Device Group. With regard to the clinical respiratory symptoms at Day 1, the improved performance of the Device Group is better than that of the Control Group (Table 5).

The power analysis was defined by Type I error 0.05 and Type II error as 0.20. In terms of the power analysis of oxygen supply, there is no difference between pre- and post-treatment data. The sample size was found to be 19 for each group compared to 50% of the four control groups. The 50 percent-sample size in four subgroups, the sample size was 19 patients for each group. When the power analysis in terms of oxygen supply (Type I error 0.05, Type II error 0.20 is taken as power) is conducted, no change has been noted between the two groups at the beginning and by the end of the treatment.

If there is a two-stage difference between the two groups before and after treatment (Type I error 0.05, Type II error 0.80 is taken as power), 34.3% in the device group and 5% in the control group, where the sample size is 25. If Type I error is defined as 0.05 and Type II as 0.80, the two-level difference between groups before and after treatment is 34.3 percent in the Device Group, and 5 percent in the Control Group, where the sample size is 25.

The reduction in the oxygen supply to correct hypoxemia in the Device Group was statistically significant compared with the Control Group (p<0.01). In the subgroup analyses based on the delivery method of the oxygen, the significance of the treatment was borderline in the nasal cannula, whereas the so-called “improvement leap” (difference in improvement) was even more pronounced for the more severe patients, who received oxygen with reservoir oxygen mask or high flow oxygen therapy (p<0.01).

### REFERENCES

1. Harenberg J, Fenyvesi T. Heparine, thrombin- and faktor-Xa-inhibitoren [Heparin, thrombin, and Factor Xa inhibitors]. Hamostaseologie. 2004 November; 24(4):261-78. German. doi: 10.1267/hamo04040261. PMID: 15526071
2. Hirsh J, Anand S S, Halperin J L, Fuster V; American Heart Association. Guide to anticoagulant therapy: Heparin: a statement for healthcare professionals from the American Heart Association. Circulation. 2001 Jun. 19; 103(24):2994-3018. doi: 10.1161/01.cir.103.24.2994. PMID: 11413093
3. Haines S T, Racine E, Aeolla M. In DiPiro J T, Talbert R L, Yee G C, Matzke G R, Wells B G, Posey L M, editors. Pharmacotherapy: a pathophysiologic approach 6th ed. New York (NY): McGraw-Hill; 2005.337-373
4. Diamant Z, Page C P. Heparin and related molecules as a new treatment for asthma. Pulm Pharmacol Ther. 2000; 13(1):1-4.
5. Matzner Y, Marx G, Drexler R, Eldor A. The inhibitory effect of heparin and related glycosaminoglycans on neutrophil chemotaxis. Thromb Haemost. 1984; 52(2):134-137.
6. Enkhbaatar P, Cox R A, Traber L D, et al. Aerosolized anticoagulants ameliorate acute lung injury in sheep after exposure to burn and smoke inhalation. Crit Care Med. 2007; 35:2805-2810.
7. Camprubi-Rimblas M, Guillamat-Prats R, Lebouvier T, Bringue J, Chimenti L, Iglesias M, Obiols C, Tijero J, Blanch L, Artigas A. Role of heparin in pulmonary cell populations in an in-vitro model of acute lung injury. Respir Res. 2017 May 10; 18(1):89. doi: 10.1186/s12931-017-0572-3.
8. Abdelaal Ahmed Mahmoud A, Mahmoud H E, Mahran M A, Khaled M. Streptokinase Versus Unfractionated Heparin Nebulization in Patients With Severe Acute Respiratory Distress Syndrome (ARDS): A Randomized Controlled Trial With Observational Controls. J Cardiothorac Vasc Anesth. 2020 February; 34(2):436-443. doi: 10.1053/j.jvca.2019.05.035. Epub 2019 May
9. Atz A M, Wessel D L. Inhaled nitric oxide and heparin for infantile primary pulmonary hypertension. Lancet 1998 June; 351(9117):1701.
10. Dixon B, Santamaria J D, Campbell D J. A phase 1 trial of nebulised heparin in acute lung injury. Crit Care 2008 May; 12:R64
11. Miller A C, Elamin E M, Suffredini A F. Inhaled anticoagulation regimens for the treatment of smoke inhaletion-associated acute lung injury: A systemic review. Crit Care Med 2014 February; 42(2):413-419.
12. Chopara A, Burkey B, Calaman S. A case report of clinically significant coagulopathy associated with aerosolized heprain and acetylcsteine therapy for inhalation injury. Burns 2011 November; 37(7):e73-5
13. Serisier D J, Shute J K, Hockey P M, Higgins B, Conway J, Carroll M P. Inhaled heparin in cystic fibrosis. Eur Respir J 2006 February; 27(2):354-8
14. Oyarzun-Ampuero F A, Brea J, Loza M I, Alonso M J, Torres D. A potential nanomedicine consisting of heparin-loaded polysaccharide nanocarriers for the treatment of asthma. Macromol Biosci 2012 February; 12(2):176-83
15. Oyarzun-Ampuero F A, Brea J, Loza M I, Torres D, Alonso M J. Chitosan-hyaluronic acid nanoparticles loaded with heparin for the treatment of asthma. Int J Pharm 2009 November; 381(2):122-9.
16. Ahmed T, Abraham W M, D'Brot J. Effects of inhaled heparin on immunologic and non immunologic bronchoconstrictor responses in sheep. Am Rev Respir Dis 1992 March; 145(3):566-70; 18.
17. Polosa R, Magri S, Vancheri C, Armato F, Santonocito G, Mistretta A, Crimi N. Time course of changes in adenosine 5′-monophosphate airway responsiveness with inhaled heparin in allergic asthma. J Allergy Clin Immunol 1997 March; 99(3):338-44.
18. Tuinman P R, Dixon B, Levi M, Juffermans N P, Schultz M J. Nebulized anticoagulants for acute lung injury—a systematic review of preclinical and clinical investigations. Crit Care 2012 Dec. 12; 16(2):R70
19. Cipolla D C, Gonda I. Formulation technology to repurpose drugs for inhalation delivery. Drug Discovery Today Therap. Strat. 2011; 8 (3-4); 123-13.
20. Wachtel, Herbert. (2016). Respiratory Drug Delivery. 10.1007/978-3-319-26920-7_9.

1. A pharmaceutical composition comprising heparin or a pharmaceutically acceptable derivative of heparin that is dissolved in a carrier solution in order to locally administer it to the lungs for use in the treatment of viral lung diseases including COVID-19 disease caused by Severe Acute Respiratory Syndrome-Coronavirus-2 (SARS-CoV-2), acute lung diseases, and/or chronic lung diseases by means of soft mist inhaler or active vibrating mesh technology nebulizer, or passive vibrating mesh technology nebulizer through inhalation route.

2. A pharmaceutical composition according to claim 1, characterized in that, heparin is low molecular weight heparin (LMWH).

3. A pharmaceutical composition according to claim 1, characterized in that, heparin is unfractionated heparin (UFH).

4. A pharmaceutical composition according to any one of claims 1-3, characterized in that, a pharmaceutically acceptable derivative of heparin is selected from heparin sodium salts, heparin esters, heparin ethers, heparin bases, heparin solvates, heparin hydrates, or forms used as heparin prodrugs.

5. A pharmaceutical composition according to any one of claims 1-4, characterized in that, the carrier solution is water for injection, water for inhalation, physiological saline (0.9% NaCl), half physiological saline (0.45% NaCl), or phosphate buffer (pH 4.5-7.4).

6. A pharmaceutical composition according to any one of claims 1-4, characterized in that, it comprises 4000-25000 IU of heparin that is dissolved in the carrier solution or a pharmaceutically acceptable derivative of heparin.

7. A pharmaceutical composition according to claim 6, characterized in that, a dose of heparin used in the treatment, or a pharmaceutically acceptable derivative of heparin is 4000 IU, 6000 IU, 8000 IU, or 10000 IU.

8. A pharmaceutical composition according to claim 7, characterized in that, a dose of heparin used in the treatment, or a pharmaceutically acceptable derivative of heparin is 4000 IU/ml, 6000 IU/ml, 8000 IU/ml, or 10000 IU/ml.

9. A pharmaceutical composition according to any one of claims 1-4, characterized in that, it further comprises at least one different active substance or at least one excipient.

10. A pharmaceutical composition according to claim 9, characterized in that, the active substance can be selected from mannitol, acetyl cysteine, or hypertonic (3-20% NaCl, w/v) physiological saline, an anti-inflammatory corticosteroid, ascorbic acid, and/or ascorbic acid derivatives.

11. A pharmaceutical composition according to claim 10, characterized in that, it is corticosteroid dexamethasone, budesonide, beclomethasone dipropionate, fluticasone, and/or mometasone.

12. A pharmaceutical composition according to claim 9, characterized in that, it comprises; at least one excipient selected from tonicity adjusting excipients, pH adjusting agents, buffering agents, tonicity adjusting agents, antioxidants, antimicrobial preservatives, surfactants, solubility enhancers (co-solvents), stabilizing agents, excipients for sustained release or prolonged local retention, wetting agents, dispensing agents, taste-masking agents, sweeteners, and/or flavor.

13. A pharmaceutical composition according to claim 12, characterized in that, co-solvent can be selected from propylene glycol, dipropylene glycol, ethylene glycol, glycerol, ethanol, polyethylene glycols, PEG300, PEG400, methanol, polyethylene glycol castor oil, polyoxyethylene castor oil, and/or lecithin.

14. A pharmaceutical composition according to claim 12, characterized in that, stabilizing agent can be selected from EDTA or its sodium salt, citric acid, sodium citrate, vitamin E, ascorbic acid, ascorbyl palmitate, butylated hydroxyanisole, butylated hydroxytoluene, hypophosphorous acid, monothioglycerol, propyl gallate, sodium bisulfite, sodium metabisulfite, sodium formaldehyde sulfoxylate, thiourea, lysine, tryptophan, phenylpropyl glycine, glycine, glutamic acid, leucine, isoleucine, serine, tea polyphenols, ascorbyl palmitate, hydroxymethyl ester, hydroxyethyl tetramethyl piperidinol, bis (2, 2,6,6-tetramethyl-4-piperidyl) sebacate, polysuccinate (4-hydroxy-2,2,6,6-tetramethyl-1-piperidinylethanol) ester, 2-[2-hydroxy-4-[3-(2-ethylhexyloxy)-2-hydroxypropoxy] phenyl]-4,6-bis (2,4-dimethylphenyl) and/or 1,3,5-triazine.

15. A pharmaceutical composition according to claim 12, characterized in that, antioxidant can be selected from primary antioxidants, reducing antioxidants and/or synergistic antioxidants.

16. A pharmaceutical composition according to claim 15, characterized in that; antioxidant can be selected from tocopherol acetate, lycopene, reduced glutathione, catalase, peroxide dismutase, acetylcysteine, R-cysteine, vitamin E TPGS, pyruvic acid and/or its magnesium or sodium salts, gluconic acid and/or its magnesium and/or sodium salts, ethylenediamine tetraacetic acid (EDTA) and/or its derivatives, ascorbic acid, esters of ascorbic acid, fumaric acid, malic acid, citric acid, butyl hydroxy anisole, butyl hydroxy toluene, propyl gallate, maltol and/or salts thereof.

17. A pharmaceutical composition according to claim 12, characterized in that, the antimicrobial preservative can be selected from quaternary ammonium compounds, thimerosal alcoholic agents, antibacterial esters, chelating agents, and/or antifungal agents.

18. A pharmaceutical composition according to claim 12, characterized in that; antimicrobial preservative can be selected from benzalkonium chloride, benzethonium chloride, cetrimide, cetylpyridinium chloride, lauralconium chloride, myristyl picolinium mercuric chloride, chlorobutanol, phenylethyl alcohol, benzyl alcohol, parahydroxybenzoic acid esters, disodium edetate (ethylenediaminetetraacetic acid, EDTA), chlorhexidine, chlorocresol, sorbic acid and/or its salts, potassium sorbate, polymyxin, sodium benzoate, sorbic acid, sodium propionate, methylparaben, ethylparaben, propylparaben, butylparaben, ethyl p-hydroxybenzoate and/or n-propyl p-hydroxybenzoate.

19. A pharmaceutical composition according to claim 12, characterized in that; pH adjusting agent can be selected from physiologically acceptable acids, bases, salts, or combinations thereof.

20. A pharmaceutical composition according to claim 12, characterized in that; pH adjusting agent can be selected from strong mineral acids, mineral bases, inorganic acids of medium-strength, organic acids of medium strength, alkaline earth hydroxides, and oxides, basic ammonium salts, carbonates, citrates.

21. A pharmaceutical composition according to claim 12, characterized in that; pH adjusting agent can be selected from sulfuric acid, hydrochloric acid, phosphoric acid, citric acid, tartaric acid, succinic acid, fumaric acid, methionine, acidic hydrogen phosphates with sodium or potassium, lactic acid, glucuronic acid, sodium hydroxide, magnesium hydroxide, calcium hydroxide, ammonium acetate, lysine, sodium carbonate, magnesium carbonate, sodium hydrogen carbonate, sodium citrate.

22. A pharmaceutical composition according to claim 12, characterized in that; the buffering agent can be selected from citric acid-sodium citrate, citric acid-phosphoric acid disodium hydrogen, potassium dihydrogen phosphate-disodium hydrogen phosphate, citric acid-sodium hydroxide, trometamol, disodium phosphate, dodecahydrate, heptahydrate, dihydrate, and anhydrous forms thereof and/or sodium mixtures.

23. A pharmaceutical composition according to claim 12, characterized in that, tonicity adjusting agent can be selected from sodium chloride, mannitol, dextrose, sodium gluconate, sodium pyruvate and/or potassium chloride, glucose, lactose, sucrose, trehalose, xylitol, sorbitol, and/or isomaltol.

24. A pharmaceutical composition according to any one of claims 1 to 4, characterized in that, it comprises a sterile isotonic saline solution to achieve desired tonicity of the dosage form.

25. A pharmaceutical composition according to claim 12, characterized in that, surfactants can be ionic or non-ionic surfactants that are safe for oral or nasal inhalation.

26. A pharmaceutical composition according to claim 25, characterized in that, the surfactant can be selected from tyloxapol, polysorbates, polysorbate 20, polysorbate 60, polysorbate 80, lecithin, vitamin E TPGS, macrogol hydroxystearates, and/or macrogol-15-hydroxystearate.

27. A pharmaceutical composition according to claim 12, characterized in that, the taste-masking agent can be selected from a group of pharmaceutically acceptable sweeteners comprising saccharin, aspartame, cyclamate, sucralose, acesulfame, neotame, thaumatin, neohesperidine, and/or salts or solvates thereof.

28. A pharmaceutical composition according to claim 12, characterized in that, the taste-masking agent can be sodium salt of saccharin or potassium salt of acesulfame.

29. A pharmaceutical composition according to claim 12, characterized in that, the taste-masking agent can be sucrose, trehalose, fructose, lactose, xylitol, mannitol, and/or isomalt.

30. A pharmaceutical composition according to claim 12, characterized in that, the taste-masking agent can be selected from pharmaceutically acceptable surfactants, alkaline earth metal salts, organic acids, and/or amino acids.

31. A pharmaceutical composition according to claim 30, characterized in that, citric acid, lactic acid, and/or arginine.

32. A pharmaceutical composition according to claim 12, characterized in that, the aromatic flavor can be selected from essential oils.

33. A pharmaceutical composition according to claim 32, characterized in that, the aromatic flavor can be menthol, thymol, or cineol.

34. A pharmaceutical composition according to claim 32, characterized in that wetting or dispensing agents can be selected from poloxamers, oleic acid or its salts, lecithin, hydrogenated lecithin, sorbitan fatty acid esters, oleyl alcohol, phospholipids including but not limited to phosphatidylglycerol, phosphatidylcholine, polyoxyethylene fatty alcohol ethers, polyoxypropylene fatty alcohol ether, polyoxyethylene fatty acid ester, glycerol fatty acid esters, glycolipid such as sphingolipid and sphingomyelin, polyoxyethylene glycol fatty acid ester, polyol fatty acid esters, polyethylene glycol glycerol fatty acid esters, polypropylene glycol fatty acid esters, ethoxylated lanolin derivatives, polyoxyethylene fatty alcohol, polyoxyethylene sorbitan fatty acid esters, polyoxyethylene stearate, propylene glycol alginate, dilauryldimethylammonium chloride, D-a-tocopheryl-PEG 1000 succinate, Polyoxy 40 stearate, polyoxyethylene-polyoxypropylene block copolymers, polyoxyethylene vegetable oils, fatty acid derivatives of amino acids, glyceride derivatives of amino acids, benzalkonium chloride and/or bile acids.

35. A pharmaceutical composition according to any one of the preceding claims for use in the treatment of COVID-19, influenza, tuberculosis, cystic fibrosis, chronic obstructive pulmonary disease (COPD), asthma, acute pulmonary infection, bronchitis, acute respiratory distress syndrome (ARDS), hypoxemia, pulmonary embolism, pulmonary hypertension, idiopathic pulmonary fibrosis, acute lung injury (ALI), sarcoidosis, and/or chronic pulmonary embolism.

36. A pharmaceutical composition according to any one of the preceding claims, characterized in that, it is single-use or multi-use dosage.

37. A pharmaceutical composition according to claim 2, characterized in that, mass median aerodynamic diameter (MMAD) value is between the range of 1-6 μm.

38. A pharmaceutical composition according to claim 2, characterized in that, mass median aerodynamic diameter (MMAD) value is 5.3 μm.

39. A pharmaceutical composition according to claim 2, characterized in that, mean fine particle fraction (FPF) value is between the range of 10-60%.

40. A pharmaceutical composition according to claim 38, mean fine particle fraction (FPF) value is 44%.

41. A pharmaceutical composition comprising low molecular weight heparin (LMWH) or a pharmaceutically acceptable derivative thereof for use in the treatment of viral lung diseases including COVID-19 disease caused by Severe Acute Respiratory Syndrome-Coronavirus-2 (SARS-CoV-2), acute lung diseases, and/or chronic lung diseases, characterized in that, it is locally administered to the lung by means of soft mist inhaler or vibrating mesh technology nebulizer through inhalation route.

42. A pharmaceutical composition comprising unfractionated heparin (UFH) or a pharmaceutically acceptable derivative thereof for use in the treatment of viral lung diseases including COVID-19 disease caused by Severe Acute Respiratory Syndrome-Coronavirus-2 (SARS-CoV-2), acute lung diseases, and/or chronic lung diseases, characterized in that, it is locally administered to the lung by means of soft mist inhaler or vibrating mesh technology nebulizer through inhalation route.

43. A pharmaceutical composition according to any one of claims 9 to 12, characterized in that, said pharmaceutical composition is in the form of emulsion or suspension.

44. A pharmaceutical composition comprising heparin or a pharmaceutically acceptable derivative of heparin that is dissolved in a carrier solution in order to locally administer it to the lungs for use in the treatment of viral lung diseases including COVID-19 disease caused by Severe Acute Respiratory Syndrome-Coronavirus-2 (SARS-CoV-2), acute lung diseases, and/or chronic lung diseases by means of soft mist inhaler or active vibrating mesh technology nebulizer, or passive vibrating mesh technology nebulizer through inhalation route.
